(19) United States
(12) Patent Application Publication
(10) Pub. No.: US 2007/0011755 A1
(43) Pub. Date: Jan. 11, 2007

(54) Diabetes type 2 animal model

(75) Inventors: Helena Edlund, Umea (SE); Michael D. Walker, Rehovot (IL); Nir Rubins, Netanya (IL); Par Steneberge, Umea (SE)

Correspondence Address: YOUNG & THOMPSON, 745 SOUTH 23RD STREET, 2ND FLOOR, ARLINGTON, VA, 22202, US

(73) Assignees: BETAGNON AB, Stockholm (SE); YEDA RESEARCH AND DEVELOPMENT CO LTD, Rehovot (IL)

(21) Appl. No.: 10/568,271

(22) PCT Filed: Aug. 18, 2004
(86) PCT No.: PCT/SE04/01209

§ 371 Date: Jun. 28, 2006

Related U.S. Application Data
(60) Provisional application No. 60/481,249, filed on Aug. 18, 2003.
(60) Provisional application No. 60/481,608, filed on Nov. 7, 2003.
(60) Provisional application No. 60/521,377, filed on Apr. 14, 2004.

(51) Int. Cl. A01K 67/027

(52) U.S. Cl. 800/14; 800/18

(57) ABSTRACT

A diabetes type 2 model laboratory animal is disclosed which comprises cells over-expressing GPR40 under the control of the Ipfl/Pdx 1 promoter. Also disclosed is the use of the model animal for the identification of agonists or antagonists towards GPR40, useful for the development of pharmaceuticals towards treatment of the disease.

### TECHNICAL FIELD

The present invention relates to an animal model, in particular a transgenic mouse over-expressing GPR40 under the control of the Ipf1/Pdx1 promoter for identification of ligands that interacts with GPR40. The mouse could be used for screening therapeutic agents influencing the GPR40 receptor and its pathways. The present invention also relates to assay systems and methods for screening of therapeutic agents. Examples of diseases involving interaction of GPR40 are diabetes, obesity, cancer (Yonezawa et al (Biochem. Biophys. Res. Commun. 2004, (314) 805-809, neurodegenerative disease (for example Alzheimer's disease, Parkinson's and Huntington's diseases) and other indications such as stroke.

Diabetes is just one of the diseases that could be treated with drugs that interfere with the GPR40 receptor and the background for Type 2 diabetes should therefore only be seen as an example to the invention.

### TECHNICAL BACKGROUND

Type 2 diabetes mellitus is a contemporary global epidemic with over 150 million people currently affected, and with an expected increase of 5-6 million cases per year. Obesity, western dietary habits, lack of physical activity and aging will continue to drive dramatic growth of type 2 diabetes for decades to come. Type 2 diabetics exhibit reduced action of insulin in skeletal muscle, fat and liver cells combined with reduced insulin secretion from the pancreatic β-cells. Type 2 diabetes develops after β-cell defects arise. Thus, by improving the adaptive β-cell response, diabetes may be treated properly. In order to find new novel treatment for Type 2 diabetes there is a need for new animal models of diabetes, namely models in which the pancreatic β-cells are defective, leading to one or more conditions or symptoms that are characteristic of diabetes. Such a model could be serving in screenings assays for the identification of agents that partially or totally compensate for the β-cell defect. Glucose is an effective and fast stimulator of insulin secretion from storage granules in β-cells. The low-affinity glucose transporter type 2, (Glut2), ensures an efficient glucose uptake by the β-cell and in the cell glucose becomes phosphorylated by the key glycolytic enzyme glucokinase. Oxidative metabolism of glucose then subsequently leads to an increase in the cytosolic ATP/ADP ratio, which in turn results in a closure of the ATP-sensitive K⁺ ATP transmembrane channel. The resulting membrane depolarisation and subsequent activation of the voltage gated Ca²⁺-channel then leads to an influx of Ca²⁺ that stimulates exocytic release of insulin and C-peptide into the bloodstream (for review see Easom et al 2000). Thus, efficient glucose uptake and subsequent metabolism ensures the generation of the secondary signals that are critical for glucose stimulated insulin secretion (GSIS). Other nutrients such as amino acids and free fatty acids (FFA) as well as hormones also influence insulin secretion but these molecules rather appear to potentiate the stimulatory effect of glucose rather than directly stimulate insulin secretion from β-cells (Rutter 2001; Zraika et al. 2002). Although fatty acids increase GSIS prolonged exposure to high concentrations of fatty acids impairs GSIS (Patane, 2003, Jean E. Schaffer. Curr Opin Lididol 2003 ). The mechanisms behind the positive and negative effects of fatty acids on GSIS remain, however, largely unknown.

Type 2 Diabetics respond inadequately to a glucose challenge, i.e. glucose intolerance diabetics and often show early signs of impaired GSIS, and loss of first-phase insulin secretion. In most cases this β-cell dysfunction progresses further, leading to complete β-cell failure and manifestation of overt diabetes in the affected individuals. The diabetic state of the individual, i.e. hyperglycemia and hyperlipidemia, are thought to contribute to the deterioration of β-cell function but the molecular mechanisms involved have remained also largely unknown (Zraika et al. 2002; Kashyap et al. 2003).

The recently identified G-protein coupled receptor GPR40 is expressed in adult β-cells (Briscoe et el 2003, Itoh et al 2003, Kotarsky et al 2003, WO-A2-02/057783). GPR40 belongs to a novel class of G-protein coupled receptors that also includes GRPR41 and GPR43 (Briscoe, Itoh, Kotarsky and Brown et al 2003) and activation of GPR40 in cell lines result in an increase in intracellular Ca²⁺ concentration and induces MAPK activity (Itoh 2003). Moreover, long-chain fatty acids enhance GSIS from insulinoma cell lines in a GPR40 dependent manner (Itoh 2003). Hence, GPR40 provides a potential candidate link between FFAs and GSIS. Lipotoxicity, i.e. perturbed β-cell function and severely impaired GSIS due to elevated levels of FFAs, becomes manifest first after long-term exposure to FFA, suggesting that over-stimulation rather than loss of function of a FFA responsive receptor with time may lead to deterioration of β-cell function, impaired GSIS, and the development of diabetes. Thus, both agonist and antagonist of GPR40 activity could be of therapeutic value for diabetes, and in particular Type 2 diabetes. Antagonist will be used for prevention of diabetes in individual with elevated levels of FFA and reduction of lipotoxicity and thus improve the function of the β-cells, and agonist for treatment of diabetes in order to stimulate GSIS. Antagonists could also be used to inhibit the proliferation of cancer cells.

### SUMMARY OF THE INVENTION

It is an object of the present invention to provide assay methods for screening of therapeutic agents that interact with the GPR40 receptor and could be used for development of novel drugs.

It is an object of the present invention to provide an animal model for screening of therapeutic agents that interact with the GPR40 receptor. The animal may be a rodent e.g. a rat or a mouse and preferably a mouse, but other laboratory animals such as monkeys, rabbits and guinea-pigs may be used.

It is an object of the present invention to provide a method for identifying agents that increase impaired insulin secretion in pancreatic β-cells by acting as a GPR40 agonist or agents acting as antagonist which will be used for prevention of diabetes in individual with elevated levels of FFA. The test agent is determined to be active as an antagonist if it increases Glut2 expression in the exposed transgenic mice, or lower the blood glucose levels, restores glucose tolerance and proinsulin processing.

It is an object of the present invention to describe compounds that antagonize the GPR40 receptor and therefore could be used to inhibit cellular proliferation for the development of cancer treatments.

It is an object of the present invention to provide an animal model which mimic human Type 2 diabetes and which can be used to develop a therapy.

Further objects of the invention will become evident from the study of the following short description of the invention and preferred embodiments thereof, the figures illustrating the invention, and the appended claims.

### DETAILED DESCRIPTION OF INVENTION

In a first aspect, the present invention provides a transgenic laboratory animal over-expressing GPR40 comprising the promotor Ipf1/Pdx1 for controlling the expression of GPR40. The transgenic animal is preferably a rodent such as a mouse or a rat, but could be another useful laboratory animal.

In a second aspect, the present invention provides a method for testing whether a chemical compound possessing a certain effect for treating diabetes Type 2 using a transgenic laboratory animal comprising the steps of:
a) providing a chemical compound to be tested;
b) providing a transgenic laboratory animal according to claim 1;
c) exposing said animal to said chemical compound; and
d) determining whether said chemical compound has an effect on the blood glucose level, triglyceride level, low density lipoprotein (LDL), high density lipoprotein (HDL), free fatty acids and/or glucose tolerance in said animal.

### DEFINITIONS

The term “chemical compound” refers to any chemical entity, pharmaceutical, drug, and the like that is suspected to affect diabetes Type 2 or cancer. Possible chemical compounds comprise both known and potential therapeutic compounds. A chemical compound can be determined to affect diabetes type 2 or cancer by exposing a transgenic animal according to the present invention to said chemical compound for a suitable amount of time, followed by monitoring known diabetes type 2 or cancer associated characteristics, such as blood glucose level, triglyceride level, low density lipoprotein (LDL), high density lipoprotein (HDL), free fatty acids and/or glucose tolerance.

A chemical compound is said to be “in a form suitable for administration such that the chemical compound is bio-available in the blood of the animal” when the chemical compound may be administered to an animal by any desired route (e.g., oral, intravenous, subcutaneous, intrathecal, intraperitoneal, intramuscular, etc.) and the chemical compound or its active metabolites appears in the blood of the animal in an active form.

Following initial screening, a chemical compound that appears promising is further evaluated by administering various concentrations of the chemical compound to the transgenic animals provided herein in order to determine an approximate therapeutic dosing range.

Animal testing may be supplemented and confirmed by testing on human subjects. However, the animal models herein provided allow the testing of a large number of compounds, both by the methods described above and other methods known in the art, in a system similar in many important respects to that in humans.

As used herein, “agent” refers to a compound, complex or substance, natural or man-made.

As used herein, “GPR40” refers to a receptor which is disclosed in for example references (Briscoe et el 2003, Itoh et al 2003, Kotarsky et al 2003, WO-A2-02/057783). GPR40 refers to a receptor polypeptide having at least 95% identity to the polypeptide sequence given in SEQ ID No: 1 and having GPR40 function. An example of the GPR40 receptor is provided in the polypeptide sequence of SEQ ID No: 1. An example of a nucleic acid sequence encoding GPR40 is disclosed as SEQ ID No:2.

As used herein, “Ipf/Pdx1” is a promotor sequence which is disclosed in reference (Apelqvist 1997).

### SUMMARY

Over-expression of GPR40 behind the Ipf1/Pdx1 promoter in mice leads to diabetes. The diabetic phenotype may in part result from loss of Glut2 expression in β-cells of the Ipf1/GPR40 mice. These data suggest that levels of GPR40 and/or activity need to be carefully controlled to assure normal β-cell function. Hence, these mice represent an important animal model that can be used to identify substances capable of modulating GPR40 activity and/or expression and thus blood glucose levels.

Test agents are screened from large libraries of synthetic or natural compounds. Numerous means are currently used for random and directed synthesis of saccharide, peptide, nucleic acid and heterocyclic based compounds. Examples of active compounds include also known GPCR binding scaffolds known for the person skilled in the art. Synthetic compound libraries are commercially available from companies such as Chembridge (Russia), Maybridge Chemical Co. (UK) Chemical Diversity (Russia), Comgenex (Hungary), Asinex (Russia) etc. Libraries of natural compounds in the form of bacterial, fungal, plant and animal extracts are available from e.g. Pan Laboratories (USA), MycoSearch (USA) or are readily producible. The agent could also be in the form of pharmaceutically acceptable salt, prodrugs.

Examples of pharmaceutically acceptable addition salts for use in the pharmaceutical compositions of the present invention include those derived from mineral acids, such as hydrochlorid, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids. The pharmaceutically acceptable excipients described herein, for example, vehicles, adjuvants, carriers or diluents, are well-known to those who are skilled in the art and are readily available to the public. The pharmaceutically acceptable carrier may be one which is chemically inert to the active compounds and which have no detrimental side effects or toxicity under the conditions of use. Pharmaceutical formulations are found e.g. in Remington: The Science and Practice of Pharmacy, 19th ed., Mack Printing Company, Easton, Pa. (1995).

Examples of prodrugs include, but are not limited to, esters and carbamates of hydroxy functional groups, esters groups of carboxyl functional groups, N-acyl derivatives, N-Mannich bases. General information on prodrugs may be found e.g. in Bundegaard, H. “Design of Prodrugs” pl-92, Elesevier, New York-Oxford (1985).

The agents according to the invention may be prepared for any route of administration, e.g. oral, intravenous, cutaneous or subcutaneous, nasal, intramuscular, or intraperitoneal. The precise nature of the carrier or other material will depend on the route of administration. For a parenteral administration, a parenterally acceptable aqueous solution is employed, which is pyrogen free and has requisite pH, isotonicity, and stability. Those skilled in the art are well able to prepare suitable solutions and numerous methods are described in the literature. A brief review of methods of drug delivery is also found in e.g. Langer, Science 249:1527-1533 (1990).

The dose administered to a mammal, particularly a human, in the context of the present invention should be sufficient to effect a therapeutic response in the mammal over a reasonable time frame. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration. In the case of oral administration the dosage can vary from about 0.01 mg to about 1000 mg per day or the corresponding amount of a pharmaceutically acceptable salt thereof.

### BRIEF DESCRIPTION OF THE DRAWINGS

FIG. 1 shows perturbed Islet morphology in IPF-1/GPR40 mice and wild type mice.

FIG. 2a shows Insulin and Glucagon double-staining in wild type mice.

FIG. 2b shows Insulin and Glucagon double-staining in IPF-1/GPR40 mice.

FIG. 3a shows Insulin and Somatostatin double staining in wild type mice.

FIG. 3b shows Insulin and Somatostatin double staining in IPF-1/GPR40 mice.

FIG. 4a shows Isl1 and PP double staining in wild type mice.

FIG. 4b shows Isl1 and PP double staining in IPF-1/GPR40 mice.

FIG. 5a,c,e,g shows the expression of factors involved in glucose sensing and proinsulin processing is perturbed in wild type mice.

FIG. 5b,d,f,h shows the expression of factors involved in glucose sensing and proinsulin processing is perturbed in Ipf1/GPR40 mice.

FIG. 6a shows insulin radio immune assay (RIA) of total pancreatic protein extracts in wild type and Ipf1/GPR40 mice.

FIG. 6b shows quantitative real time PCR of insulin mRNA from isolated islet cDNA of wild type and Ipf1/GPR40 mice.

FIG. 7a shows the glucose tolerance test of Ipf1/GPR40 and wild type mice.

FIG. 7b shows the insulin secretion in response to glucose challenge of Ipf1/GPR40 and wild type mice.

FIG. 8a,b shows impaired insulin secretion in Ipf1/GPR40 transgenic mice where the serum insulin levels were determined in wild type and Ipf1/GPR40 mice after intraperitoneal injection of glucose into over-night fasted animals.

FIG. 9 shows a map on construct.

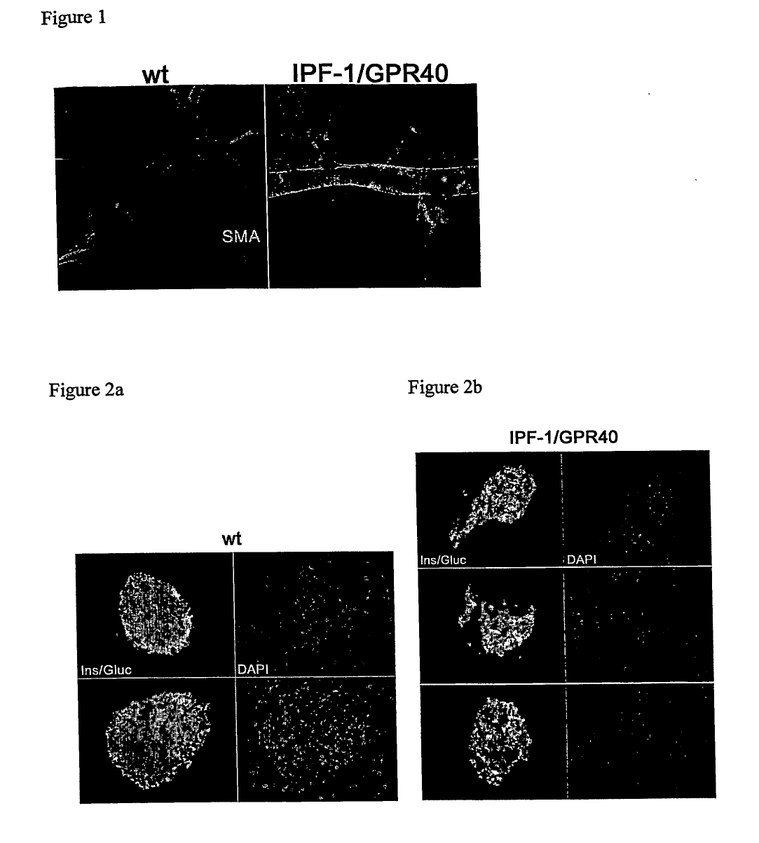

Several diseases could be treated by compounds which interact with the GPR40 receptor and in order to investigate the function of GPR40 in vivo the inventors have generated transgenic mice carrying over-expressing GPR40 β-cells under the control of the Ipf1/Pdx1 promoter (Apelqvist 1997), see FIG. 1. This animal model could be used for studies of Type 2 diabetes. Other animal models could be developed in the same approach for the studies of other diseases. The resulting transgenic mice carrying over-expressing GPR40 β-cells were born alive and appeared initially healthy but developed diabetes with age, see Table 1, suggesting that forced expression of GPR40 in adult β-cells perturb β-cell function. The results, as described below, from the transgenic mice point to new ways in which manipulation of the expression of the GPR40 lead to diabetes and could be used for studying novel therapeutic agents for the treatment of diabetes.

GPR40 transgene expression in early (e10) pancreatic progenitor cells (right panel).

TABLE 1

Mean conc. of Blood glucose (mmol/l)

| | 9 days | 21 days | 32 days | 40 days |
|---|---|---|---|---|
| Wt | 3.8 (1) | 8.1 (9) | 9.3 (9) | 9.3 (8) |
| Ipf-1/GPR40 | 7.2 (6) | 11.9 (9) | 19.3 (9) | 16.4 (8) |

Blood glucose levels are elevated in Ipf-1/GPR40 transgenic as compared to wild type littermates as shown in Table 1.

Pancreases

Analyses of isolated pancreases 10 weeks old mice showed no apparent difference in overall organ size between transgenic and wild type littermates (not shown). Whole mount immunostaining of the pancreases derived from transgenic and wild-type mice using antibodies against Insulin, Glucagon and alpha-Smooth Muscle Actin revealed a normal organisation of major blood vessels and a normal distribution of clustered pancreatic endocrine cells, i.e. islets, in the transgenic mice, see FIG. 1. Nevertheless, the intercellular organization of endocrine cells in the islets appeared perturbed and the islets of the transgenic mice lack the normal organization of glucagon expressing α-cells surrounding a core of tightly clustered β-cells and instead the glucagon expressing α-cells appear interspersed within the islets of Ipf-1/GPR40 mice. Hence, the more purple colour of islets in transgenic mice, see FIG. 1, right panel. Perturbed Islet morphology in IPF-1/GPR40 mice.

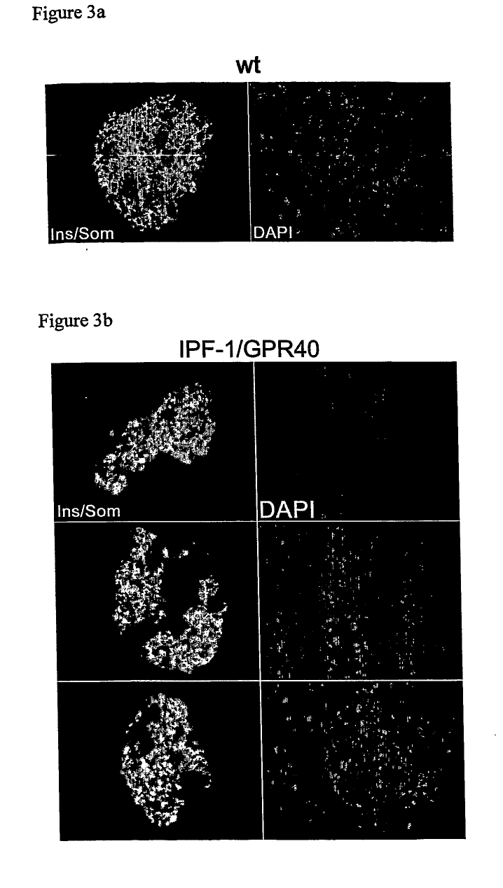

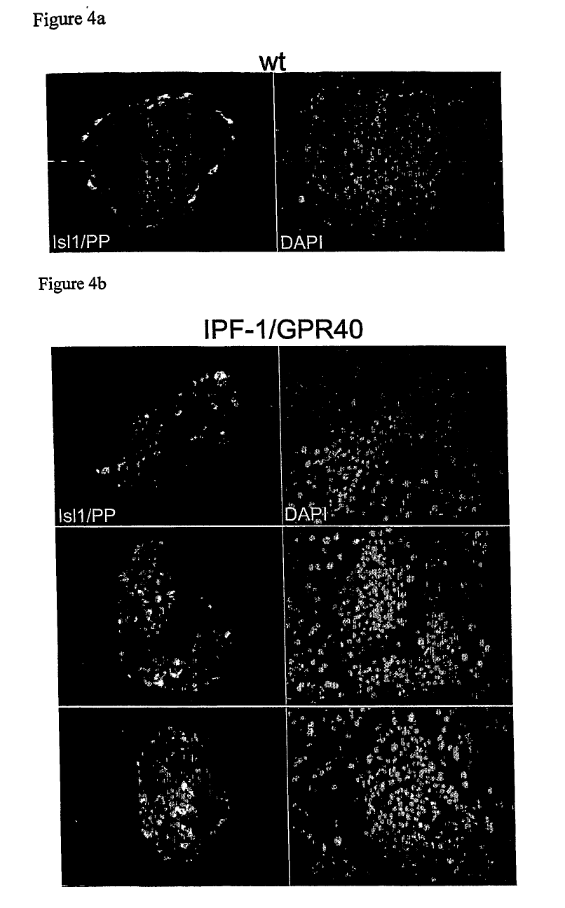

Immunostaining of sectioned pancreas derived from wild type and transgenic mice using antibodies directed against insulin, glucagons, somatostatin, Isl1 and pancreatic polypeptide (PP) confirmed the abnormal organisation of the endocrine cells within the islets of transgenic mice, see FIGS. 2b, 3b and 4b. FIG. 2a,b shows insulin and Glucagon double-staining. FIG. 3a,b shows insulin and Somatostatin double staining and FIG. 4a,b shows Isl1 and PP double staining.

Molecular Defects

To begin to identify the molecular defects underlying the development of diabetes in the Ipf1/GPR40 mice analysis of the expression of factors known to be critically required for β-cell function where performed.

Glut 2 and PC1/3

Long-term exposure of islets to elevated FFA in vitro results in an impaired expression of factors controlling β-cell function (Gremlich et al 1997, Zhou et al 2003). One such factor is Glut2, the expression of which is also perturbed in a variety of diabetic animal models (Efrat 2003). In the Ipf1/GPR40 mice the expression of Glut2 was virtually undetectable providing evidence that Glut2 expression is down-regulated in Ipf1/GPR40 mice, see FIG. 5a,b.

Another factor is the prohormone convertase 1/3 (PC1/3), one of the key enzymes involved in proinsulin to insulin processing, The expression of PC1/3 was reduced in the Ipf1/GPR40 mice, see FIG. 5c,d which give an increased proinsulin-to-insulin (P/I) ratio. The increase ratio is associated with type 2 diabetes. The proinsulin immunoreactivity was readily detectable in β-cells of the Ipf1/GPR40 mice see, FIG. 5e,f. The reduced expression of Glut2 and PC1/3 will lead to perturbed glucose sensing and proinsulin processing in β-cells of Ipf1/GPR40 mice and are likely to contribute to the impaired GSIS and development of diabetes observed in these mice. This was confirmed by immunohistochemical analyses using anti-proinsulin specific antibodies that revealed an increased amount of proinsulin in β-cells of Ipf1/GPR40 mice as compared to wild type mice, see FIG. 5e,f. These data suggest that GPR40 expression and/or activity in adult β-cells need to be tightly balanced to ensure normal GSIS and hence glucose homeostasis.

FIG. 5 shows the expression of factors involved in glucose sensing and proinsulin processing is perturbed in Ipf1/GPR40 mice. a-d, Analyses of Glut2 (a,b) and PC1/3 (c,d) expression show that Glut2 expression is impaired and PC1/3 expression reduced in β-cells of Ipf1/GPR40 mice. e-g, Proinsulin processing is perturbed in Ipf1/GPR40 mice and hence in addition to insulin (g,h), proinsulin (e,f) immunoreactivity is readily observed in β-cells of the Ipf1/GPR40 mice.

Proinsulin and Insulin

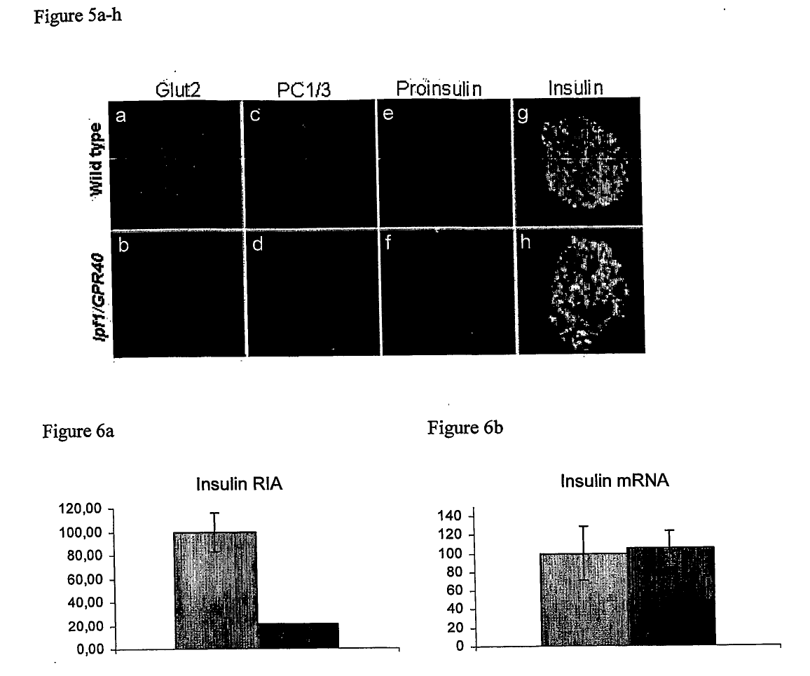

Hypersecretion of insulin and proinsulin followed by hypoinsulinaemia is associated with the progression of type 2 diabetes as mentioned above and long-term exposure of islets to elevated levels of FFAs also leads to increased secretion, predominantly of proinsulin, and depletion of insulin content (Furukawa et al., Diabetes 48,1395-1401, 1999; Bjorklund & Grill, Diabetes, 48, 1409-1414, 1999). The total pancreatic insulin content was severely reduced in the Ipf1/GPR40 mice, see FIG. 6a, suggesting perturbed insulin gene expression and or/insulin storage in Ipf1/GPR40 mice. The impaired GSIS observed in the Ipf1/GPR40 mice may hence, at least in part, result from a shortage of ready releasable insulin. Real-time PCR analyses of insulin mRNA levels revealed that insulin gene expression was normal in Ipf1/GPR40 mice, see FIG. 6b. Taken together these data show that pancreatic development, β-cell generation, and insulin gene is expression normal but that pancreatic insulin content is reduced in Ipf1/GPR40 mice, resembling the condition observed in type 2 diabetics and islets exposed elevated FFAs.

Reduced pancreatic insulin content but normal insulin gene expression in Ipf1/GPr40 mice, see FIGS. 6a and 6b. FIG. 6a shows insulin radio immune assay (RIA) of total pancreatic protein extracts, showing that the total pancreatic insulin content is reduced by ˜78% in Ipf1/GPR40 mice (blue) as compared to wild type controls (pink). FIG. 6b shows, quantitative real time PCR of insulin mRNA from isolated islet cDNA of wild type (blue, n=3) and Ipf1/GPR40 (pink, n=3) mice showing that insulin gene expression is normal in the Ipf1/GPR40 mice.

Glucose Tolerance Test

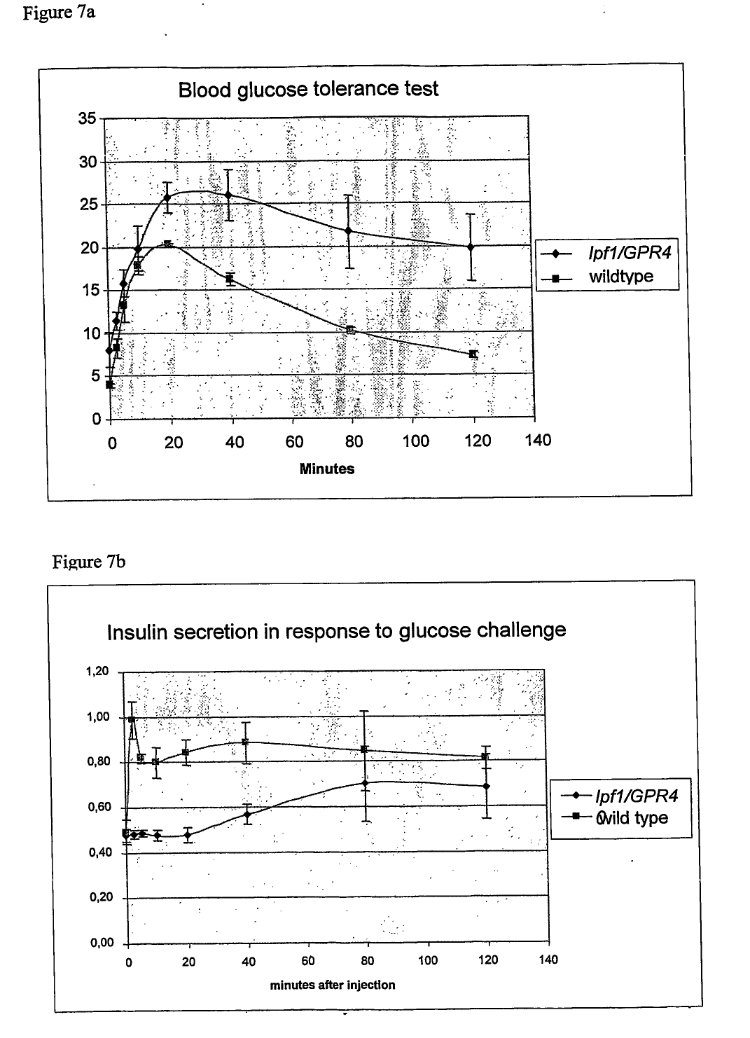

Glucose tolerance test (Ulf Ahigren et. Al. 1998) and determination of GSIS by measuring serum insulin levels in response to exogenous glucose to overnight fasted mice revealed a severely impaired glucose tolerance in these mice, a loss of first-phase insulin release and a delayed, blunted second phase insulin release in the Ipf1/GPR40 mice as compared to the wild-type controls, see FIG. 7a and FIG. 7b.

FIG. 7a shows that the Ipf1/GPR40 mice are glucose intolerant. Glucose tolerance test of Ipf1/GPR40 (♦,n-4) and wild type mice (▪,n=2) showing blood glucose levels (shown as mmol/l on the x-axis) after intraperitoneal (i.p.) injection of glucose into over-night fasted mice. Average values. FIG. 7b shows the impaired insulin secretion in Ipf1/GPR40 transgenic mice. Serum insulin levels (shown as ng/ml on the x-axis) were determined in wild type (▪, n=2) and Ipf1/GPR40 (♦, n=4) mice after i.p. injection of glucose into overnight fasted animals. Average values.

Free Fatty Acids

A human cell surface receptor is activated by free fatty acids and thiazolidinedione drugs. Measurements of free fatty acids is disclosed in references (Knut Kotarsky 2003 and Jean E. Schaffer 2003).

Measurements of triglyceride level, low density lipoprotein (LDL) level and high density lipoprotein (HDL) level is known to the skilled person in the art. Some measurement methods are disclosed in U.S. Pat. No. 4,125,377, U.S. Pat. No. 4,920,123 and U.S. Pat. No. 6,764,828.

UCP2, PPAR, Cpg21, MKP 3 and CREM-17X

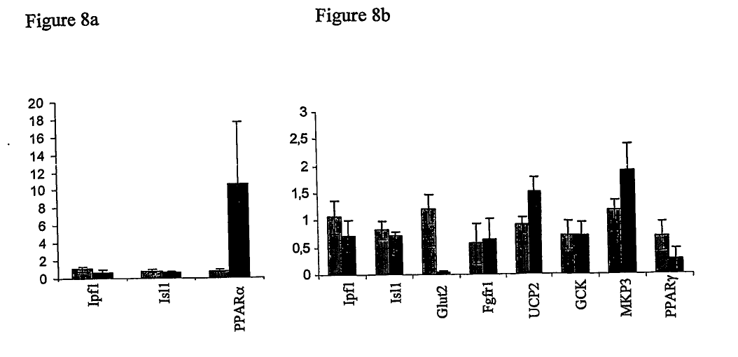

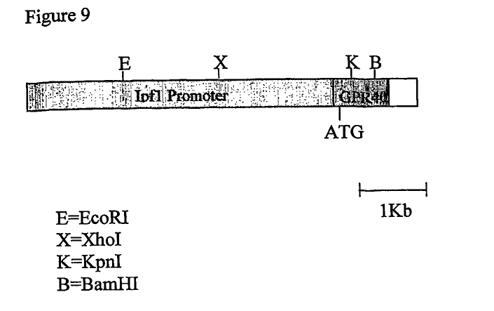

A number of different factors, including the dual-specific MAPK-phosphatase Cpg21, cAMP response element modulator (CREM)-17X, the peroxisome proliferator-activated (PPAR) α, and its downstream target genes carnitine palmitoyltransferase 1 (CPT1) and uncoupling protein 2 (UCP2,) are all up-regulated rather then down regulated in islets exposed to long-term FFAs (Zraika et al 2002, Zhou 2003). Real-time PCR analysis showed that the expression of PPARα, UCP2, and the dual-specificity MAPK phosphatases (MKP) 3 were up-regulated in Ipf1/GPR40 mice, whereas the expression of PPARγ, a gene linked to type 2 diabetes, was reduced, see FIG. 8a,b. The expression of CPT1 was up-regulated 6-fold in Ipf1/GPR40 mice. The virtually abolished expression of Glut2 was confirmed on the mRNA level, see FIG. 8a,b. A number of genes, including the transcription factors Ipf1/Pdx1 and Isl1, the fibroblast growth factor receptor (Fgfr) 1, and glucokinase (GCK), Cpg21, and CREM-17X, showed unchanged or moderately altered expression levels (FIG. 8a,b and data not shown).

FIG. 8a,b shows quantitative real time PCR on islet cDNA from wild type (pink, n=3) and Ipf1/GPR40 (blue, n=3) mice, demonstrating that Glut2 expression is virtually abolished, PPARγ reduced, UCP2, MKP3 and PPARα expression increased.

cp-2 is known to impair GSIS by uncoupling respiration from oxidative phosphorylation, which in turn leads to reduced ATP-production. The exact role of PPARs in β-cell function is poorly understood but PPARs are fatty acid sensors that regulate genes involved in oxidation and lipogenesis. PPARα have been shown to stimulate UCP2 expression and impair insulin secretion in an insulinoma cell line (Tordjman et al 2002). Attenuation of FGFR1c signalling in mouse β-cells leads to diabetes, partly due to impaired expression of Glut2 and PC1/3 (Hart et al 2000), and FGF signalling is known to activate the MEK/MAPK (Szebenyi et al 1999). Dual-specificity phospatases, such as MKP3, are in turn known to dephosphorylate and inactivate ERK, leading to inhibition of MAPK-dependent FGF-signalling (Keyse, 2000). The up-regulation of MKP3 expression observed in the Ipf1/GPR40 mice would thus impair the FGFR1c signalling in β-cells. Together these data demonstrate that forced expression of GPR40 in adult β-cells in vivo perturbs the expression of factors required for appropriate glucose sensing, oxidative metabolism, and proinsulin to insulin processing.

Generation of Ipf1/GPR40 Transgenic Mice

The GPR40 coding ORF was isolated as a 930-basepair XbaI-BgIII restriction fragment from clone E7 (Michael Walker). Following fill in of 5′-overhang the isolated fragment was cloned behind the Ipf1/Pdx1 promotor (Apelqvist, 1997). Transgenic mice were generated by pronuclear injection of a purified 6.5-kb NotI-BssHII restriction-fragment encompassing the Ipf1/Pdx1 promotor followed by the GPR40 cDNA into F2 hybrid oocytes from B6/CBA parents as described (Hogan B. Manipulating the mouse embryo, 1994). Genomic DNA extracted from tail biopsies or embryonic heads were used in PCR analyses to determine the genotype of transgenic animals. The primers used were: 5′-GGGAAGAGGAGATGTAGACTT-3′ (Ipf1/Pdx1 primer for 5′) and 5′-GTAGAGGGGAGCAAAGTG-3′ (GPR40 primer 3′). Expression from the transgene was confirmed by in situ staining on e10 embryos.

The promotor Islet amyloid (IAPP) (Hull et. Al. 2004), could also be used to generate transgenic mice over-expressing GPR40 in beta cells.

Glucose and Insulin Measurements

All functional analyses were performed in vivo using overnight fasted mice that were injected intraperitoneally with the Glucose (1 g/kg) body weight. Blood samples were obtained from the tail vein before glucose injection (time 0 min) and at 2.5, 5, 10, 20, 40, 80 and 120 minutes after glucose injection. Blood glucose levels were measured using a Glucometer Elite (Bayer Inc.) and serum insulin levels were measured using ELISA (Mercodia) according to the manufacturers recommendations.

Quantification of mRNA Expression Levels

cDNA was prepared from total RNA prepared from isolated islets (Ahren, 1997) using NucleoSpin RNAII-kit (#635990, Machery-Nagel) and Super SMART PCR (#635000, Clontech). Real time PCR analysis was performed using the ABI PRISM 7000 Sequence Detection System and SYBR Green PCR Master Mix (ABI) according to the manufacturers recommendations.

In Situ Hybridizations and Immunohistochemistry

In Situ hybridisations using a DIG-labelled probe corresponding to mouse GPR40 cDNA was carried out as described (Apelqvist, 1997). Immunohistochemical localization of antigens and double-label immunohistochemistry was carried out as described (Apelqvist, 1997). Primary antibodies used were: guniea pig-anti-Insulin (Linco), rabbit-anti-Glucagon (EuroDiagnostica), rabbit-anti-Glucose transporter 2 (kindly provided by B. Thorens, Lausanne, Switzerland), rabbit-anti-Prohormone convertase 1/3 (Chemicon), rat-anti-Somaostatin (Biogenesis), guinea pig-anti-Pancreatic polypeptide (Linco), rabbit-anti-Islet1 (kindly provided by T. Edlund, Umeå, Sweden). Secondary antibodies used were: ALEXA 488-anti-guniea pig (Molecular Probe), Cy3-anti-rabbit and Cy3-anti-rat (Jackson laboratory).

### REFERENCES

Ahren B, Simonsson E, Scheurink A J, Mulder H, Myrsen U et al. Dissociated insulinotropic sensitivity to glucose and carbachol in high-fat diet-induced insulin resistance in C57BL/6J mice. Metabolism 46: 97-106 (1997).
Apelqvist A, Ahlgren U, Edlund H. Sonic hedgehog directs specialised mesoderm differentiation in the intestine and pancreas. Curr Biol 7:801-8044 (1997).
Briscoe C P, Tadayyon M, Andrews J L, Benson W G, Chambers J K, Eilert M M, Ellis C, Elshourbagy N A, Goetz A S, Minnick D T, Murdock P R, Sauls H R Jr, Shabon U, Spinage L D, Strum J C, Szekeres P G, Tan K B, Way J M, Ignar D M, Wilson S, Muir A I. The orphan G protein-coupled receptor GPR40 is activated by medium and long chain fatty acids. J Biol Chem. 278:11303-11311 (2003).
Brown A J, Goldsworthy S M, Barnes M, Eilert M M, Tcheang L, Daniels D, Muir A I, Wigglesworth M J, Kinghorn I, Fraser N J, Pike N B, Strum J C, Steplewski K M, Murdock P R, Holder J C, Marshall F H, Szekeres P G, Wilson S, Ignar D M, Foord S M, Wise A, Dowell S J. The Orphan G protein-coupled receptors GPR41 and GPR43 are activated by propionate and other short chain carboxylic acids. J Biol Chem. 278:11312-11319 (2003).
Bjorklund, A., & Grill, V. Enhancing effects of long-term elevated glucose and palmitate on stored and secreted proinsulin-to-insulin ratios in human pancreatic islets. Diabetes, 48:1409-14 (1999).
Easom R A. Beta-granule transport and exocytosis. Semin Cell Dev Biol 11, 253-66 (2000).
Furukawa H, Carroll R J, Swift H H, Steiner D F. Long-term elevation of free fatty acids leads to delayed processing of proinsulin and prohormone convertases 2 and 3 in the pancreatic beta-cell line MIN6. Diabetes, 48:1395-1401 (1999).
Gremlich S, Bonny C, Waeber G, Thorens B. Fatty acids decrease IDX-1 expression in rat pancreatic islets and reduce GLUT2, glucokinase, insulin, and somatostatin levels. J Biol Chem.; 272:30261-30269 (1997).
Hart A W, Baeza N, Apelqvist A, Edlund H. Attenuation of FGF signalling in mouse beta-cells leads to diabetes. Nature 408(6814): 864-868. (2000).
Hogan, B., Beddington, R., Costantini, F. & Lacy, E n. Manipulating the Mouse Embryo. Cold Spring Harbour Laboratorey Press, 1994.
Itoh Y, Kawamata Y, Harada M, Kobayashi M, Fujii R, Fukusumi S, Ogi K, Hosoya M, Tanaka Y, Uejima H, Tanaka H, Maruyama M, Satoh R, Okubo S, Kizawa H, Komatsu H, Matsumura F, Noguchi Y, Shinohara T, Hinuma S, Fujisawa Y, Fujino M. Free fatty acids regulate insulin secretion from pancreatic beta cells through GPR40. Nature 422:173-176 (2003).
Kashyap S, Belfort R, Gastaldelli A; Pratipanawatr T, Berria R, Pratipanawatr W, Bajaj M, Mandarino L, DeFronzo R, Cusi K. A sustained increase in plasma free Fatty acids impairs insulin secretion in nondiabetic subjects genetically predisposed to develop type 2 diabetes. Diabetes 52:2461-2474 (2003).
Keyse S M. Protein phosphatases and the regulation of mitogen-activated protein kinase signalling. Curr Opin Cell Biol. 12:186-192 (2000).
Kotarsky K, Nilsson N E, Flodgren E, Owman C, Olde B. Kotarsky K, Nilsson N E, Flodgren E, Owman C, Olde B. A human cell surface receptor activated by free fatty acids and thiazolidinedione drugs. Biochem Biophys Res Commun. 301:406-410 (2003).
Patane G, Anello M, Piro S, Vigneri R, Purrello F, Rabuazzo A M. Role of ATP production and uncoupling protein-2 in the insulin secretory defect induced by chronic exposure to high glucose or free fatty acids and effects of peroxisome proliferator-activated receptor-gamma inhibition. Diabetes 51:2749-2756 (2002).
Rutter G A. Nutrient-secretion coupling in the pancreatic islet β-cell: recent advances. Mol Aspects of Medicine 22, 247-284 (2001).
Szebenyi G, Fallon J F. Fibroblast growth factors as multifunctional signaling factors. Int Rev Cytol 185: 45-106 (1999).
Tordjman K, Standley K N, Bernal-Mizrachi C, Leone T C, Coleman T, Kelly D P, Semenkovich C F. PPARalpha suppresses insulin secretion and induces UCP2 in insulinoma cells. J Lipid Res. 43:936-943 (2002).
Zhou Y P, Marlen K, Palma J F, Schweitzer A, Reilly L, Gregoire F M, Xu G G, Blume J E, Johnson J D. Overexpression of repressive cAMP response element modulators in high glucose and fatty acid-treated rat islets. A common mechanism for glucose toxicity and lipotoxicity? J Biol Chem. 278:51316-51323 (2003).
Zraika, S, Dunlop, M, Proietto, J, Anrikopoulos. Effects of free fatty acids on insulin secretion in obesity. Obesity reviews, 3:103-112, 2002.
Yonezawa T, Katoh K, Obara Y Existence of GPR40 functioning in a human breast cancer cell line, MCF-7. Biochem Biophys Res Commun. 314:805-809 (2004).
Hull R L, Westermark G T, Westermark P, Kahn S E. Islet amyloid: a critical entity in the pathogenesis of type 2 diabetes. J Clin Endocrinol Metab. August 2004; 89(8):3629-43.
β-Cell-specific inactivation of the mouse Ipf1/Pdx1 gene results in loss of the β-cell phenotype and maturity onset diabetes.
Ulf Ahigren, Jörgen Jonsson, Lena Jonsson, Karin Simu, and Helena Edlund. Genes and Development 1998 12:1763-1768.
Type 2 Diabetes: An overview. Harold Lebovitz
Clinical Chemistry 1999 45:8(B), 1339-1345.
Knut Kotarsky, Niclas E. Nilsson, Erik Flodgren, Christer Owman, and Björn Olde. Biochemical and Biophysical Research Communications 301 (2003) 406-410.
Lipotoxicity: When tissues overeat. Jean E. Schaffer. Curr Opin Lididol 2003 14:281-287.

1-9. (canceled)

10. A transgenic non-human laboratory animal over-expressing GPR40 comprising the promoter lpf1/Pdx1 for controlling the expression of GPR40.

11. The transgenic animal of claim 10, wherein the animal is a rodent.

12. The transgenic animal of claim 11, wherein the animal is a mouse or a rat.

13. A method for testing whether a chemical compound possessing a certain effect for treating diabetes Type 2 using a transgenic laboratory animal comprising the steps of:
a) providing a chemical compound to be tested;
b) providing a transgenic laboratory animal according to claim 10;
c) exposing said animal to said chemical compound; and
d) determining whether said chemical compound has an effect on the blood glucose level in said animal.

14. A method for testing whether a chemical compound possessing a certain effect for treating diabetes Type 2 using a transgenic laboratory animal comprising the steps of:
a) providing a chemical compound to be tested;
b) providing a transgenic laboratory animal according to claim 10;
c) exposing said animal to said chemical compound; and
d) determining whether said chemical compound has an effect on the triglyceride level in said animal.

15. A method for testing whether a chemical compound possessing a certain effect for treating diabetes Type 2 using a transgenic laboratory animal comprising the steps of:
a) providing a chemical compound to be tested;
b) providing a transgenic laboratory animal according to claim 10;
c) exposing said animal to said chemical compound; and
d) determining whether said chemical compound has an effect on the low density lipoprotein (LDL) level in said animal.

16. A method for testing whether a chemical compound possessing a certain effect for treating diabetes Type 2 using a transgenic laboratory animal comprising the steps of:
a) providing a chemical compound to be tested;
b) providing a transgenic laboratory animal according to claim 10;
c) exposing said animal to said chemical compound; and
d) determining whether said chemical compound has an effect on the high density lipoprotein (HDL) level in said animal.

17. A method for testing whether a chemical compound possessing a certain effect for treating diabetes Type 2 using a transgenic laboratory animal comprising the steps of:
a) providing a chemical compound to be tested;
b) providing a transgenic laboratory animal according to claim 10;
c) exposing said animal to said chemical compound; and
d) determining whether said chemical compound has an effect on the free fatty acids in said animal.

18. A method for testing whether a chemical compound possessing a certain effect for treating diabetes Type 2 using a transgenic laboratory animal comprising the steps of:
a) providing a chemical compound to be tested;
b) providing a transgenic laboratory animal according to claim 10;
c) exposing said animal to said chemical compound; and
d) determining whether said chemical compound has an effect on the glucose tolerance content in said animal.


SEQUENCE LISTING (4 sequences)

SEQ ID NO: 1 - protein, 300 aa, Mus musculus
MDLPPQFSFALYVSAFALGFPLNLLAIRGAVSHAKLRLTPSLVYTLHLGCSDLLLAITLPLKAVEALASGAWPLPLPFCPVFALAHFAPLYAGGGFLAALSAGRYLGAAFPFGYQAIRRPRYSWGVCVAIWALVLCHLGLALGLETSGSWLDNSTSSLGINIPVNGSPVCLEAWDPDSARPARLSFSILLFFLPLVITAFCYVGCLRALVRSGLSHKRKLRAAWVAGGALLTLLLCLGPYNASNVASFINPDLGGSWRKLGLITGAWSVVLNPLVTGYLGTGPGRGTICVTRTQRGTIQK

SEQ ID NO: 2 - DNA, 903 bp, Mus musculus
atggacctgcccccacagttctccttcgctctctatgtatctgcctttgcgctgggctttccattgaacttgttagccatccgaggcgcagtgtcccacgctaaactgcgactcactcccagcttggtctacactctccatctgggctgctctgatctcctactggccatcactctgcccctgaaggctgtggaggccctggcttctggagcctggcccctgccgctccccttctgcccagtctttgccttggcccactttgctcccctctacgcaggcggaggcttcctagctgctctcagcgctggccgctacctgggggctgccttccccttcgggtaccaagccatccggaggccccgctattcctggggtgtgtgtgtggctatatgggcccttgtcctctgccacctggggctggcccttggcttggagacttccggaagctggctggacaacagtaccagttccctgggcatcaacatacccgtgaatggctccccggtctgcctggaagcctgggatcccgactctgcccgccctgcccgtctcagtttctccattctgctcttctttctgcccttggtcatcactgccttctgctatgtgggctgcctccgggccctggtgcgctcaggcctgagccacaaacggaagctcagggcagcttgggtggccggaggcgctctcctcacactcctgctctgcctggggccctataatgcctccaatgtggctagtttcataaacccggacctaggaggctcctggaggaagttgggactcatcacaggggcctggagtgtggtactcaacccactggtcactggctacttgggaacaggtcctggacggggaacaatatgtgtgacgaggactcaaagaggaacaattcagaagtag

SEQ ID NO: 3 - DNA, 21 bp, Artificial Sequence
  Description of Artificial Sequence Synthetic primer
gggaagaggagatgtagactt

SEQ ID NO: 4 - DNA, 18 bp, Artificial Sequence
  Description of Artificial Sequence Synthetic primer
gtagaggggagcaaagtg